(19) United States
(12) Patent Application Publication
(10) Pub. No.: US 2020/0085641 A1
(43) Pub. Date: Mar. 19, 2020

(54) ABSORBENT ARTICLE WITH IMPROVED LEG AND FLAP ELASTICS

(71) Applicant: Kimberly-Clark Worldwide, Inc., Neenah, WI (US)

(72) Inventor: Sarah Kleuskens, Neenah, WI (US)

(21) Appl. No.: 16/471,828

(22) PCT Filed: Dec. 20, 2017
(86) PCT No.: PCT/US17/67591

§ 371 Date: Jun. 20, 2019

Related U.S. Application Data
(60) Provisional application No. 62/440,865, filed on Dec. 30, 2016.

(51) Int. Cl. A61F 13/496; A61F 13/494; A61F 13/49; A61F 13/15

(52) U.S. Cl. CPC A61F 13/496; A61F 13/15585; A61F 13/49009; A61F 13/4942

(57) ABSTRACT

Absorbent articles with improved barrier and leg cuffs are disclosed. In an embodiment, an absorbent article comprises a chassis and a combined leg and barrier cuff coupled to the chassis which comprises leg elastic members, at least one barrier cuff elastic member, and a cuff material encompassing all of the elastic members. The first combined leg cuff and barrier cuff further comprises a first un-elasticized portion extending between a seam bond and a leg elastic member, a first elasticized portion encompassing the leg elastic members, and a second un-elasticized portion extending between the seam bond and the barrier cuff elastic member. A length encompassing the first un-elasticized portion and the first elasticized portion is greater than the length of the second un-elasticized portion, and the length of the first un-elasticized portion is less than or equal to the length of the second un-elasticized portion.

### CROSS-REFERENCE TO RELATED APPLICATION

This application claims the benefit of U.S. Provisional Application No. 62/440865, filed Dec. 30, 2016.

### TECHNICAL FIELD

The present disclosure relates to absorbent articles, and more particularly to absorbent articles with improved flap and leg elastics.

### BACKGROUND OF THE DISCLOSURE

A primary function of personal care absorbent articles is to absorb and retain body exudates such as urine, fecal material, blood, and menses with additional desired attributes including low leakage of the exudates from the absorbent article and a dry feel to the wearer of the absorbent article. By preventing leakage of the exudates from the absorbent article, the absorbent article intends to prevent the body exudates from soiling or contaminating a wearer's or caregiver's clothing or other articles, such as bedding, that can come in contact with the wearer.

One way in which absorbent articles retain and absorb exudates is with elasticated leg cuffs and containment flaps. The leg cuffs and containment flaps may each form a gasket against the wearer's skin in order to help retain exudates within the absorbent article where they can be absorbed into the interior of the absorbent article. Absorbent articles with enhanced abilities to retain and absorb exudates are continually desired to help prevent soiling of clothing or other articles.

### SUMMARY OF THE DISCLOSURE

The absorbent articles disclosed herein are designed to reduce leakage of exudates outside of the articles. In general, the absorbent articles disclosed herein are constructed with an improved elasticated leg and flap laminate piece designed to help prevent leakage. More specifically, the elasticated leg and flap laminate piece is constructed with the elastics of both the leg cuff and the containment flap bonded as part of a single structure and attached to the absorbent article. The laminate piece may be bonded to the article such that at least one of the leg elastics is disposed outboard of a side edge of the absorbent article. Additionally, the dimensions of the leg and flap laminate piece are chosen to ensure proper gasketing of both the leg cuff and containment flap to help prevent leakage, as will be explained in greater detail below.

In a first embodiment, an absorbent may comprise a chassis having a front waist region and a rear waist region, the front waist region and the rear waist region being joined by a crotch region, the chassis further having a body facing surface and a garment facing surface, the chassis extending in a longitudinal direction and having a first longitudinally extending side edge and a longitudinally extending lateral side edge. The chassis may comprise a bodyside liner, a garment facing outer cover, and an absorbent body disposed between the bodyside liner and the outer cover, and a first combined leg cuff and barrier cuff structure bonded to the body facing surface of the chassis proximate the first longitudinally extending side edge and extending between the front waist region and the rear waist region. The first combined leg cuff and barrier cuff structure may comprise a plurality of leg elastic members, at least one of the plurality of leg elastic members disposed outboard of the first longitudinally extending side edge of the chassis, at least one barrier cuff elastic member, and a cuff material encompassing the plurality of leg elastic members and the at least one barrier cuff elastic member. The first combined leg cuff and barrier cuff structure may be bonded to the chassis by a seam bond between the cuff material and the chassis, the seam bond being located between the plurality of leg elastic members and the at least one barrier cuff elastic member and dividing the combined leg cuff and barrier cuff structure into a leg cuff portion and a barrier cuff portion. Additionally, the first combined leg cuff and barrier cuff structure further may comprise a first un-elasticized portion extending between the seam bond and the leg elastic member of the plurality of leg elastic members most proximate to the seam bond, a first elasticized portion extending between the leg elastic member most proximate to the seam bond and a distal end of the leg cuff portion, and a second un-elasticized portion extending between the seam bond and the at least one barrier cuff elastic member that is most proximate to the seam bond. Finally, the length of the first un-elasticized portion plus the length of the first elasticized portion may be greater than the length of the second un-elasticized portion, and the length of the first un-elasticized portion may be less than or equal to the length of the second un-elasticized portion.

In a second embodiment, the cuff material of the first embodiment may comprise a single piece of material wrapped around the plurality of leg elastic members and the at least one barrier cuff elastic member.

In a third embodiment, the seam bond, of the second embodiment, between the first combined leg cuff and barrier cuff structure and the body facing surface of the chassis may further bond a first end of the single piece of material with a second end of the single piece of material.

In a fourth embodiment, a plurality of the plurality of leg elastic members of any of the first through third embodiments may be disposed outboard of the first longitudinally extending side edge of the chassis.

In a fifth embodiment, the at least one barrier cuff elastic member of any of the first through fourth embodiments may comprise a plurality of barrier cuff elastic members.

In a sixth embodiment, the length of the first un-elasticized portion of any of the first through fifth embodiments may be less than the length of the second un-elasticized portion by between about 5% and about 50%.

In a seventh embodiment, the length of the first un-elasticized portion of any of the first through sixth embodiments may be less than the length of the second un-elasticized portion by between about 10% and about 50%.

In an eighth embodiment, the length of the first un-elasticized portion of any of the first through seventh embodiments may be less than the length of the second un-elasticized portion by between about 15% and about 50%.

In a ninth embodiment, the length of the first un-elasticized portion of any of the first through eighth embodiments may be less than the length of the second un-elasticized portion by between about 15% and about 45%.

In a tenth embodiment, the length of the first un-elasticized portion of any of the first through ninth embodiments may be less than the length of the second un-elasticized portion by between about 20% and about 45%.

In an eleventh embodiment, a method of forming an absorbent article may comprise moving an absorbent article chassis in a machine direction, the chassis having a front waist region and a rear waist region, the front waist region and the rear waist region being joined by a crotch region, the chassis further having a body facing surface and a garment facing surface, the chassis extending in a longitudinal direction and having a first longitudinally extending side edge and a second longitudinally extending side edge, and moving a first combined leg cuff and barrier cuff structure in the machine direction. The first combined leg cuff and barrier cuff structure may comprise a plurality of leg elastic members, at least one barrier cuff elastic member, and a cuff material encompassing the plurality of leg elastic members and the at least one barrier cuff elastic member. The method may further comprise bonding the first combined leg cuff and barrier cuff structure to the body facing surface of the chassis with a seam bond between the cuff material and the body facing surface of the chassis such that at least one of the plurality of leg elastic members is disposed outboard of the first lateral side edge of the chassis. Additionally, the seam bond may divide the first combined leg cuff and barrier cuff structure into a first leg cuff and a first barrier cuff, the first leg cuff having a first leg cuff un-elasticized portion extending between the seam bond and the leg elastic member of the plurality of leg elastic members most proximate to the seam bond, the first leg cuff having a first leg cuff elasticized portion extending between the leg elastic member most proximate to the seam bond and a distal end of the first leg cuff, and the first barrier cuff having a first barrier cuff un-elasticized portion extending between the seam bond and the at least one barrier cuff elastic member that is most proximate to the seam bond. Finally, a length of the leg cuff may be greater than a length of the barrier cuff, and a length of the first leg cuff un-elasticized portion may be less than or equal to a length of first barrier cuff un-elasticized portion.

In a twelfth embodiment, the length of the first leg cuff un-elasticized portion of the eleventh embodiment may be less than the length of the first barrier cuff un-elasticized portion by between about 5% and about 50%.

In a thirteenth embodiment, the length of the first leg cuff un-elasticized portion of the eleventh embodiment or the twelfth embodiment may be less than the length of the first barrier cuff un-elasticized portion by between about 10% and about 50%.

In a fourteenth embodiment, the length of the first leg cuff un-elasticized portion of any of the eleventh through thirteenth embodiments may be less than the length of the first barrier cuff un-elasticized portion by between about 15% and about 50%.

In a fifteenth embodiment, the length of the first leg cuff un-elasticized portion of any of the eleventh through fourteenth embodiments may be less than the length of the first barrier cuff un-elasticized portion by between about 15% and about 45%.

In a sixteenth embodiment, the length of the first leg cuff un-elasticized portion of any of the eleventh through fifteenth embodiments may be less than the length of the first barrier cuff un-elasticized portion by between about 20% and about 45%.

The above summary of the present disclosure is not intended to describe each embodiment or every implementation of the present disclosure. Advantages and attainments, together with a more complete understanding of the disclosure, will become apparent and appreciated by referring to the following detailed description and claims taken in conjunction with the accompanying drawings.

### BRIEF DESCRIPTION OF DRAWINGS

The disclosure may be more completely understood in consideration of the following detailed description of various embodiments in connection with the accompanying drawings, in which:

FIG. 1 perspective view of an absorbent article according to aspects of the present disclosure;

FIG. 1A perspective view of a different absorbent article according to aspects of the present disclosure;

FIG. 2 is a top plan view of the absorbent article of FIG. 1 in a stretched, laid flat and unattached condition and including a connecting portion, according to aspects of the present disclosure;

FIG. 2A is a top plan view of the absorbent article of FIG. 1A in a stretched, laid flat and unfastened condition and including a connecting portion, according to aspects of the present disclosure; and

FIG. 3 is a cross-section of the absorbent article of FIG. 2 taken along line 3-3.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION OF THE DISCLOSURE

The present disclosure is generally directed toward absorbent articles constructed with leg elastics and containment flap elastics combined into a single laminate structure, and where the single laminate structure is bonded to the articles such that at least one of the leg elastics is disposed outboard of a side edge of the chassis of the absorbent article. Additionally, the single laminate structure has dimensions that result in proper gasketing of the containment flaps against the skin of the wearer in order to help prevent leakage of bodily exudates.

Each example is provided by way of explanation and is not meant as a limitation. For example, features illustrated or described as part of one embodiment or figure can be used on another embodiment or figure to yield yet another embodiment. It is intended that the present disclosure include such modifications and variations.

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

Definitions:

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, adult diapers and pants, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded”, “attached” or “coupled” refers herein to the joining, adhering, connecting, attaching, or the like, of two elements. Two elements will be considered bonded, attached or coupled together when they are joined, adhered, connected, attached, or the like, directly to one another or indirectly to one another, such as when each is directly bonded to intermediate elements. The bonding, attaching or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer liquids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, hydroentangling processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “stretch film laminate” refers herein to a laminate of an elastic film laminated to at least one outer layer of fibrous nonwoven web material. Desirably, the elastic film is air and vapor permeable and liquid impermeable and elastic in at least one and preferably two directions generally perpendicular to one another. More desirably, the elastic film is elastic in all directions of the X-Y plane of the material. In other embodiments, the elastic film can have cover layers, such as fibrous nonwoven web materials laminated to both sides of the elastic film so as so form a laminate with opposed exterior nonwoven layers, an intermediate layer of elastic film bonded to both of the exterior nonwoven layers. Further information can be found with reference to U.S. Pat. No. 7,803,244 to Siqueira et al. and U.S. Pat. No. 8,361,913 to Siqueira et al., each of which is incorporated herein in its entirety by reference.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

Absorbent Article:

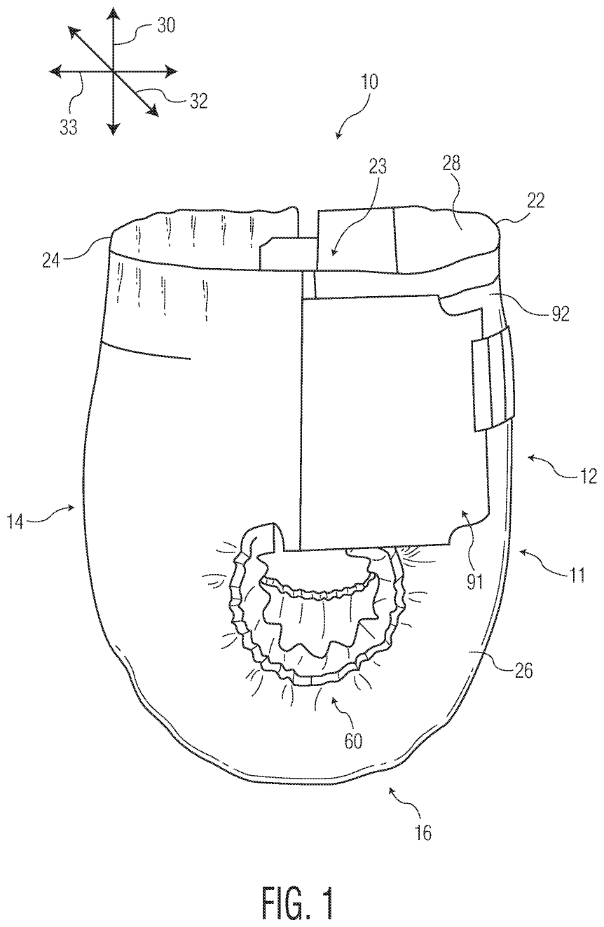

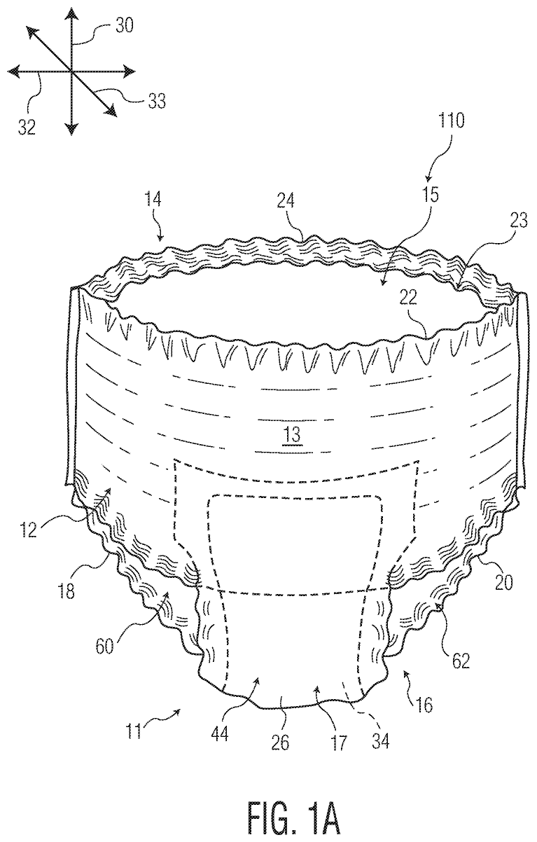

FIGS. 1-4 depict exemplary absorbent articles according to the present disclosure. One of the features of the articles of the present disclosure is the unique configuration of the combined leg cuff and containment flap laminate structures, in combination with their positioning on the exemplary absorbent articles. Specifically, the absorbent articles of the present disclosure include combined leg cuff and BM containment flap (also referred to herein as simply “containment flaps” or “flaps” or “BM flaps”) structures which include the elastic strands of both of the leg cuffs and the containment flaps in a single laminate structure. Additionally, the combined laminate structures are bonded to the absorbent articles in a specific configuration where at least one of the elastic strands of the leg cuffs is disposed outboard of the chassis of the absorbent articles. The laminate structure is also bonded to the absorbent article chassis along a portion of the laminate structure that divides the laminate structure into the leg cuff portion and the containment flap portion, with each of the portions having dimensions that allow for proper fit and gasketing of the articles and the containment flaps of the articles to help prevent leakage.

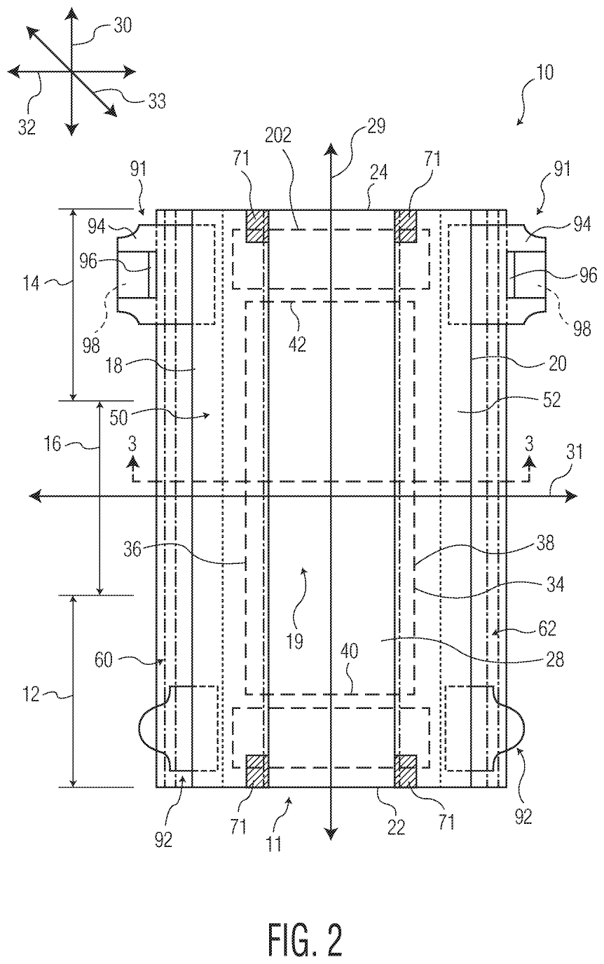

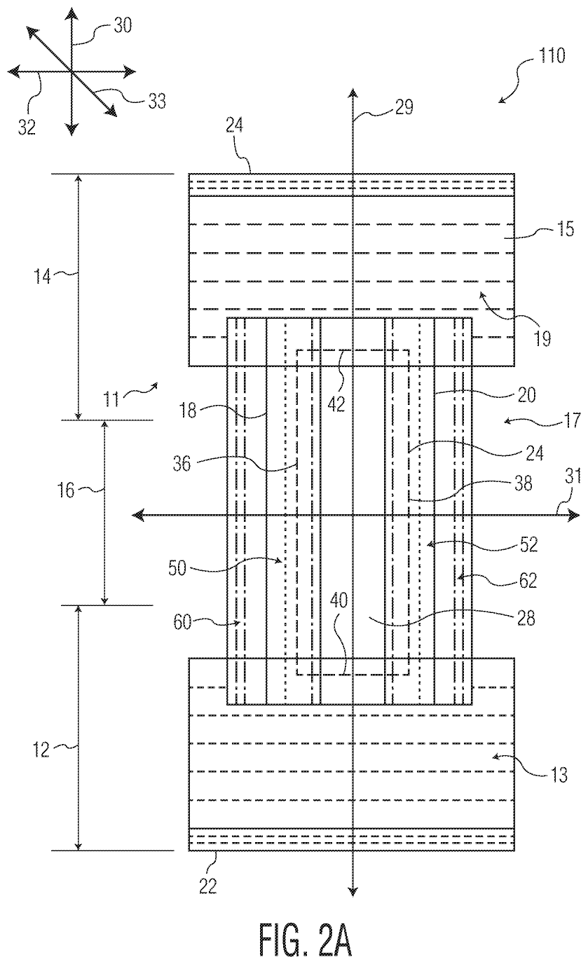

Referring to FIGS. 1-4, a non-limiting illustration of an absorbent article 10 for example, a diaper, is illustrated. Other embodiments of the absorbent article 10 can include, but are not limited to, training pants, youth pants, adult incontinence garments, and feminine hygiene articles. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure. For example, the absorbent article 110 in FIGS. 1A and 2A provides an exemplary embodiment of an absorbent article 110 that can be manufactured in cross-direction manufacturing process.

The absorbent article 10 illustrated in FIGS. 1-4 can include a chassis 11. The absorbent article 10 can further include a front waist region 12 and a rear waist region 14, and a crotch region 16 disposed between the front waist region 12 and the rear waist region 14 and interconnecting the front and rear waist regions, 12, 14, respectively. In the embodiment depicted in FIGS. 1A and 2A, a three-piece construction of an absorbent article 110 is depicted where the absorbent article 110 can have a chassis 11 including a front waist panel 13 defining the front waist region 12, a rear waist panel 15 defining the rear waist region 14, and an absorbent panel 17 defining the crotch region 16 of the absorbent article 110. The absorbent panel 17 can extend between the front waist panel 13 and the rear waist panel 15. In some embodiments, the absorbent panel 17 can overlap the front waist panel 13 and the rear waist panel 15. The absorbent panel 17 can be bonded to the front waist panel 13 and the rear waist panel 15 to define a three-piece construction. However, it is contemplated that an absorbent article can be manufactured in a cross-direction without being a three-piece construction garment which is also sometimes referred to as a one-piece construction (not shown) as the front waist panel 13 and the rear waist panel 15 are integral with one another by way of commonly connected components forming the waist panel such as a bodyside liner and/or an outer cover which can envelope the absorbent panel 17 or simply cover the garment side of the absorbent panel 17.

The absorbent article 10, 110 can have a pair of longitudinally extending side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinally extending side edges 18, 20 can extend from the front waist edge 22 to the rear waist edge 24. The longitudinally extending side edges 18, 20 can extend in a direction parallel to the longitudinal direction 30 for their entire length, such as for the absorbent article 10 illustrated in FIG. 2. In other embodiments, the longitudinally extending side edges 18, 20 can be curved between the front waist edge 22 and the rear waist edge 24. In the absorbent article 110 of FIGS. 1A and 2A, the longitudinally extending side edges 18, 20 can include portions of the front waist panel 13, the absorbent panel 17, and the rear waist panel 15.

The front waist region 12 can include the portion of the absorbent article 10, 110 that, when worn, is positioned at least in part on the front of the wearer while the rear waist region 14 can include the portion of the absorbent article 10, 110 that, when worn, is positioned at least in part on the back of the wearer. The crotch region 16 of the absorbent article 10, 110 can include the portion of the absorbent article 10, 110 that, when worn, is positioned between the legs of the wearer and can partially cover the lower torso of the wearer. Dimensionally, the front waist region 12 can be defined as the front one-third of the overall longitudinal dimension of the absorbent article 10 measuring from the front waist edge 22 to the rear waist edge 24. The rear waist region 14 can be defined as the rear one-third of the longitudinal dimension of the absorbent article 10 and the crotch region 16 is the middle one-third of the longitudinal dimension of the overall absorbent article 10. The waist edges, 22 and 24, of the absorbent article 10, 110 are configured to encircle the waist of the wearer and together define a central waist opening 23 (as labeled in FIG. 1 and FIG. 1A) for the waist of the wearer. Portions of the longitudinally extending side edges 18, 20 in the crotch region 16 can generally define leg openings for the legs of the wearer when the absorbent article 10, 110 is worn.

The absorbent article 10, 110 can include an outer cover 26 and a bodyside liner 28. The outer cover 26 and the bodyside liner 28 can form a portion of the chassis 11 where the outer cover is disposed at the garment facing surface of the chassis (not shown; opposite body facing surface 19 of chassis 11) and the bodyside liner 28 is disposed at the body facing surface 19 of the chassis 11. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIGS. 2 and 2A, the absorbent article 10, 110 can have a longitudinal axis 29 extending in the longitudinal direction 30, and a lateral axis 31 extending in the lateral direction 32. The lateral axis 31 is located midway between the front waist edge 22 and the rear waist edge 24. The absorbent article 10 also has a vertical or z-direction component extending in the vertical direction 33.

The chassis 11 can include an absorbent body 34. The absorbent body 34 can be disposed between the outer cover 26 and the bodyside liner 28. The absorbent body 34 can have longitudinal edges, 36 and 38, which, in an embodiment, can form portions of the longitudinally extending side edges, 18 and 20, respectively, of the absorbent article 10, 110. The absorbent body 34 can have a first end edge 40 that is opposite a second end edge 42, respectively, which, in an embodiment, can form portions of the waist edges, 22 and 24, respectively, of the absorbent article 10. In some embodiments, the first end edge 40 can be in the front waist region 12. In some embodiments, the second end edge 42 can be in the rear waist region 14. In an embodiment, the absorbent body 34 can have a length and width that are the same as or less than the length and width of the absorbent article 10, 110. The bodyside liner 28, the outer cover 26, and the absorbent body 34 can form part of an absorbent assembly 44. In the absorbent article 110 of FIGS. 1A and 2A, the absorbent panel 17 can form the absorbent assembly 44. The absorbent assembly 44 can also include a fluid transfer layer 46 and a fluid acquisition layer (not shown) between the bodyside liner 28 and the fluid transfer layer 46 as is known in the art. The absorbent assembly 44 can also include a spacer layer 48 disposed between the absorbent body 34 and the outer cover 26.

The absorbent article 10, 110 can be configured to contain and/or absorb liquid, solid, and semi-solid body exudates discharged from the wearer. To this end, the absorbent article 10, 110 is fitted with a pair of containment flaps 50, 52 which are configured to provide a barrier to the lateral flow of body exudates. In some further embodiments, the absorbent article 10, 110 can optionally include a waist containment member, such as waistband 202 depicted in dashed lines which cooperates with the containment flaps 50, 52 to form a pocket to further retain body exudates. See, for example, WO2016/159981 filed 31 Mar. 2015; WO2016/159978 filed 31 Mar. 2015; WO2016/159983 filed 31 Mar. 2015; WO2017/003423 filed 29 Jun. 2015; WO2017/039599 filed 31 Aug. 2015; and 62/212051, Attorney Docket Number 64968524US01 filed 31 Aug. 2015 each of which is incorporated herein by reference in its entirety.

Outer cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10, 110. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein and it also may be apertured to enhance evaporation of urine in the event the inner layer is vapor permeable.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid body exudates from leaking out of the absorbent article 10, 110 and wetting articles, such as bed sheets and clothing, as well as the wearer and caregiver.

In some embodiments, where the outer cover 26 is of a single layer construction, it can be embossed and/or matte finished to provide a more cloth-like texture or appearance. The outer cover 26 can permit vapors to escape from the absorbent article 10, 110 while preventing liquids from passing through. A suitable liquid impermeable, vapor permeable material can be composed of a microporous polymer film or a non-woven material which has been coated or otherwise treated to impart a desired level of liquid impermeability.

Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10, 110. The absorbent body 34 can have a length and width that can be less than or equal to the length and width of the absorbent article 10, 110.

In an embodiment, the absorbent body 34 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 34 can be a matrix of cellulosic fluff and superabsorbent material. In an embodiment, the absorbent body 34 may be constructed of a single layer of materials, or in the alternative, may be constructed of two or more layers of materials.

Various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent body 34 can be free of superabsorbent material or in an alternate embodiment be comprised entirely of superabsorbent material.

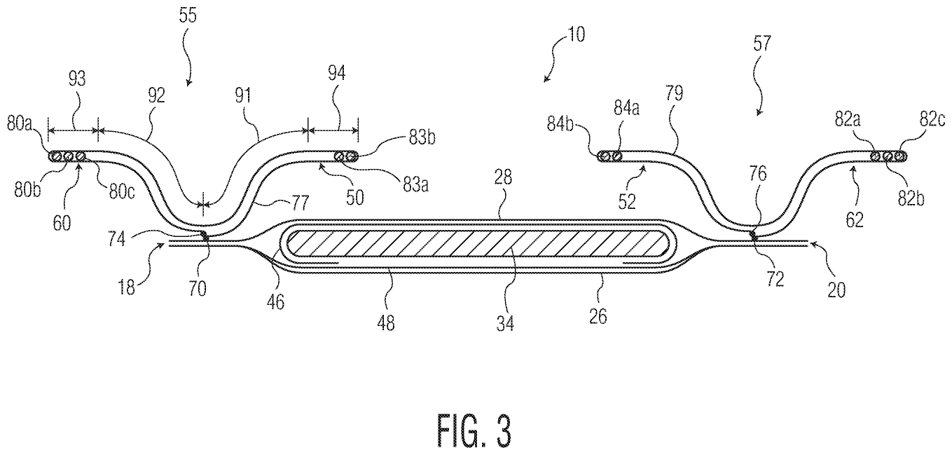

If a spacer layer 48 is present, the absorbent body 34 can be disposed on the spacer layer 48 and superposed over the outer cover 26. The spacer layer 48 can be bonded to the outer cover 26, for example, by adhesive. In some embodiments, a spacer layer 48 may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In some embodiments, at least a portion of a layer, such as but not limited to, a fluid transfer layer 46 and/or a spacer layer 48, can be positioned between the absorbent body 34 and the outer cover 26, such as illustrated in FIG. 3. The absorbent body 34 can be bonded to the fluid transfer layer 46 and/or the spacer layer 48. Typically the absorbent body 34 will be completely enveloped by a core wrap material such as a tissue wrap or a nonwoven material such a meltblown web, a spunbond web or both.

Bodyside liner:

The bodyside liner 28 of the absorbent article 10, 110 can overlay the absorbent body 34 and the outer cover 26 and can isolate the wearer's skin from liquid waste retained by the absorbent body 34. In various embodiments, a fluid transfer layer 46 can be positioned between the bodyside liner 28 and the absorbent body 34. In various embodiments, an acquisition layer (not shown) can be positioned between the bodyside liner 28 and the absorbent body 34 or a fluid transfer layer 46, if present. In various embodiments, the bodyside liner 28 can be bonded to the acquisition layer, or to the fluid transfer layer 46 if no acquisition layer is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 34 and/or a fluid transfer layer 46, if present, and/or an acquisition layer, if present, and/or a spacer layer 48, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. It is contemplated that the bodyside liner 28 may be narrower than the outer cover 26. However, in other embodiments, the bodyside liner 28 and the outer cover 26 may be of the same dimensions in width and length, for example, as depicted in the embodiments illustrated in FIG. 1. In other embodiments, the bodyside liner 28 can be of greater width than the outer cover 26. It is also contemplated that the bodyside liner 28 may not extend beyond the absorbent body 34 and/or may not be secured to the outer cover 26. In some embodiments, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material such as a central region of material which is different from one or both of the lateral regions of the bodyside liner 28 (not shown). The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,327,473 to Kirby et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from about 10 or 12 to about 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10, 110. In other aspects, the bodyside liner 28 can be stretchable, and more suitably elastic, in both the lateral and the longitudinal directions 32, 30, respectively.

Containment Flaps and Leg Cuffs:

As can be seen in FIG. 3, article 10 may include combined leg cuff and containment flap laminate structures 55, 57, which contain both elastic strands 83a-b, 84a-b of containment flaps 50, 52 and elastic strands 80a-c, 82a-c of leg cuffs 60, 62. In such embodiments, laminate structures 55, 57 may comprise single pieces of cuff material 77, 79 which may be wrapped around all of elastic strands 80a-c, 82a-c, 83a-b, and 84a-b. Ends of the single pieces of cuff material 77, 79 may be bonded together by bonds 74, 76 in order to form closed laminate structures. However, it should be understood this is only one contemplated configuration. In other embodiments, each of laminate structures 55, 57 may comprise multiple pieces of cuff material with elastic strands 80a-c, 82a-c, 83a-b, and 84a-b positioned between the multiple pieces of material. In such embodiments, the pieces of material may be bonded together to enclose the elastic strands 80a-c, 82a-c, 83a-b, and 84a-b between the multiple pieces of cuff material.

The laminate structures 55, 57 may further be bonded to chassis 11. In at least some embodiments, laminate structures 55, 57 may be bonded to chassis 11 such that one or more of leg elastic strands 80a-c, 82a-c are disposed outboard of the side edges 18, 20 of chassis 11. In the embodiment of FIG. 3, all of leg elastic strands 80a-c, 82a-c are disposed outboard of side edges 18, 20. However, in other contemplated embodiments, any number of leg elastic strands 80a-c, 82a-c may be disposed outboard of side edges 18, 20 of chassis 11, including as few as one of leg elastic strands 80a-c, 82a-c of each of laminate structures 55, 57.

Laminate structures 55, 57 may be bonded to chassis 11 through seam bonds 70, 72. Seam bonds 70, 72 may be formed by any suitable bonding method, including using adhesives, ultrasonic energy, heat energy, pressure, or any combination of such methods. Additionally, although seam bonds 70, 72 are shown aligned with bonds 74, 76, this is not required. In other embodiments, bonds 74, 76 may be located outboard of seam bonds 70, 72 in some embodiments or inboard of seam bonds 70, 72 in other embodiments. Seam bonds 70, 72 may divide laminate structures 55, 57 into containment flap portions 50, 52 and leg cuff portions 60, 62.

Accordingly, each of leg cuffs 60, 62 may have leg cuff lengths which comprise both un-elasticized leg cuff portion length 92 and elasticized leg cuff portion length 93 (only shown with respect to cuff 60 in FIG. 3). Each of containment flap 50, 52 may have containment flap lengths which comprise both un-elasticized containment flap portion length 91 and elasticized containment flap portion length 94. Un-elasticized leg cuff portion lengths 92 may generally span from seam bonds 70, 72 up to the leg elastic member most proximal to seam bonds 70, 72—e.g. leg elastic members 80c, 82a in FIG. 3. Elasticized leg cuff portion length 93 may generally span between the leg elastic member most proximal to seam bonds 70, 72 and the distal ends of leg cuffs 60, 62. Likewise, un-elasticized containment flap portion lengths 91 may generally span from seam bonds 70, 72 up to the flap elastic member most proximal to seam bonds 70, 72—e.g. flap elastic members 83a, 84a in FIG. 3. Elasticized containment flap portion lengths 94 may generally span between the flap elastic member most proximal to seam bonds 70, 72 and the distal ends of the containment flaps 50, 52.

In order for the containment flaps 50, 52 and the leg cuffs 60, 62 to gasket a wearer in an optimal fashion, it is important that the portions of the laminate structures 55, 57 have particular dimensions in relation to other portions of the laminate structures 55, 57. For example, in some embodiments, the length of leg cuffs 60, 62, that is un-elasticized leg cuff portion length 92 plus elasticized leg cuff portion length 93, may be greater than un-elasticized containment flap portion length 91. In some more particular embodiments, the un-elasticized leg cuff portion length 92 may be less than or equal to the un-elasticized containment flap portion length 91. In still more particular embodiments, the un-elasticized leg cuff portion length 92 may be less than the un-elasticized containment flap portion length 91 by between about 5% and about 50%, or between about 10% and 50%, or between about 15% and about 50%, or between about 20% and about 50%, or between about 20% and about 45%, or between about 25% and about 45%, or between about 30% and about 40%.

In at least some embodiments, it may be additionally beneficial for the un-elasticized containment flap portion length 91 to be greater than or equal to elasticized leg cuff portion length 93. In this way, the laminate structures 55, 57 of the present disclosure ensure that the flap elastic strands 83a-b, 84a-b are able to reach a wearer's skin when article 10 is worn by a wearer and thus provide effective gasketing.

In general, suitable elastic materials for the flap elastic strands 83a-b, 84a-b can include, but are not limited to, spandex elastomeric strands, strands of natural or synthetic rubber, thermoplastic elastomeric materials, or heat activated elastomeric materials. The flap elastic strands 83a-b, 84a-b can be any elastomeric material capable of being elongated at least about 50 percent, desirably about 350 percent, and capable of recovering to within at least about 250 percent, and desirably about 150 percent of its original length after being elongated about 300 percent. The flap elastic strands 83a-b, 84a-b can be a spandex elastomeric strand(s), or polyurethane-based synthetic fibers, such as LYCRA. Alternatively, the flap elastic strands 83a-b, 84a-b can be composed of a thermoplastic elastomer or a natural or a synthetic rubber, or even a heat activated elastic material, which can be activated with heat treatment after the flap elastic strands 83a-b, 84a-b, and laminate structures 55, 57, are secured to the chassis 11. In at least some embodiments, the elastomeric strands may have diameters that range between about 10 denier to about 1500 denier.

During manufacture of the laminate structures 55, 57 at least a portion of the flap elastic strands 83a-b, 84a-b can be bonded to the one or more pieces of material 77, 79 while the flap elastic strands 83a-b, 84a-b are elongated. The percent elongation of the flap elastic strands 83a-b, 84a-b can be, for example, about 110% to about 350%. The flap elastic strands 83a-b, 84a-b can be coated with adhesive while elongated to a specified length prior to attaching the flap elastic strands 83a-b, 84a-b within the laminate structures 55, 57. In a stretched condition, the length of the flap elastic strands 83a-b, 84a-b which have adhesive coupled thereto can provide an active flap elastic region, which will gather upon relaxation of the absorbent article 10. The active flap elastic region can be of a longitudinal length that is less than the length of the absorbent article 10, 110. In this exemplary method of bonding the flap elastic strands 83a-b, 84a-b within the laminate structures 55, 57, the portion of the flap elastic strands 83a-b, 84a-b not coated with adhesive will retract after the flap elastic strands 83a-b, 84a-b and the absorbent article 10 are cut in manufacturing to form an individual absorbent article 10. As noted above, the relaxing of the flap elastic strands 83a-b, 84a-b in the active flap elastic region when the absorbent article 10, 110 is in a relaxed condition can cause each laminate structures 55, 57 to gather and cause portions of each of the containment flaps 50, 52 to extend away from the body facing surface 19 of the chassis 11.

Additionally, for suitable elastic materials may be used for the leg elastic strands 80a-c, 82a-c. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate.

Fastening System:

In an embodiment, the absorbent article 10, can include a fastening system. The fastening system can include one or more back fasteners 91 and one or more front fasteners 92. The embodiments being shown in FIGS. 1 and 2 depict embodiments with one front fastener 92. Portions of the fastening system may be included in the front waist region 12, rear waist region 14, or both. The fastening system can be configured to secure the absorbent article 10 about the waist of the wearer in a fastened condition as shown in FIG. 1 and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 2.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

Those skilled in the art will recognize that the present disclosure may be manifested in a variety of forms other than the specific embodiments described and contemplated herein. Specifically, the various features described with respect to the various embodiments and figures should not be construed to be applicable to only those embodiments and/or figures. Rather, each described feature may be combined with any other feature in various contemplated embodiments, either with or without any of the other features described in conjunction with those features. Accordingly, departure in form and detail may be made without departing from the scope of the present disclosure as described in the appended claims.

What is claimed is:

1. An absorbent article comprising:
a chassis having a front waist region and a rear waist region, the front waist region and the rear waist region being joined by a crotch region, the chassis further having a body facing surface and a garment facing surface, the chassis extending in a longitudinal direction and having a first longitudinally extending side edge and a longitudinally extending lateral side edge, the chassis comprising:
a bodyside liner,
a garment facing outer cover, and
an absorbent body disposed between the bodyside liner and the outer cover; and
a first combined leg cuff and barrier cuff structure bonded to the body facing surface of the chassis proximate the first longitudinally extending side edge and extending between the front waist region and the rear waist region, the first combined leg cuff and barrier cuff structure comprising:
a plurality of leg elastic members, at least one of the plurality of leg elastic members disposed outboard of the first longitudinally extending side edge of the chassis;
at least one barrier cuff elastic member; and
a cuff material encompassing the plurality of leg elastic members and the at least one barrier cuff elastic member,
wherein the first combined leg cuff and barrier cuff structure is bonded to the chassis by a seam bond between the cuff material and the chassis, the seam bond being located between the plurality of leg elastic members and the at least one barrier cuff elastic member and dividing the combined leg cuff and barrier cuff structure into a leg cuff portion and a barrier cuff portion, and
the first combined leg cuff and barrier cuff structure further comprising:
a first un-elasticized portion extending between the seam bond and the leg elastic member of the plurality of leg elastic members most proximate to the seam bond;
a first elasticized portion extending between the leg elastic member most proximate to the seam bond and a distal end of the leg cuff portion; and
a second un-elasticized portion extending between the seam bond and the at least one barrier cuff elastic member that is most proximate to the seam bond,
wherein the length of the first un-elasticized portion plus the length of the first elasticized portion is greater than the length of the second un-elasticized portion, and
wherein the length of the first un-elasticized portion is less than or equal to the length of the second un-elasticized portion.

2. The article of claim 1, wherein the cuff material comprises a single piece of material wrapped around the plurality of leg elastic members and the at least one barrier cuff elastic member.

3. The article of claim 2, wherein the seam bond between the first combined leg cuff and barrier cuff structure and the body facing surface of the chassis further bonds a first end of the single piece of material with a second end of the single piece of material.

4. The article of claim 1, wherein a plurality of the plurality of leg elastic members are disposed outboard of the first longitudinally extending side edge of the chassis.

5. The article of claim 1, wherein the at least one barrier cuff elastic member comprises a plurality of barrier cuff elastic members.

6. The article of claim 1, wherein the length of the first un-elasticized portion is less than the length of the second un-elasticized portion by between about 5% and about 50%.

7. The article of claim 1, wherein the length of the first un-elasticized portion is less than the length of the second un-elasticized portion by between about 10% and about 50%.

8. The article of claim 1, wherein the length of the first un-elasticized portion is less than the length of the second un-elasticized portion by between about 15% and about 50%.

9. The article of claim 1, wherein the length of the first un-elasticized portion is less than the length of the second un-elasticized portion by between about 15% and about 45%.

10. The article of claim 1, wherein the length of the first un-elasticized portion is less than the length of the second un-elasticized portion by between about 20% and about 45%.

11. A method of forming an absorbent article, comprising:
moving an absorbent article chassis in a machine direction, the chassis having a front waist region and a rear waist region, the front waist region and the rear waist region being joined by a crotch region, the chassis further having a body facing surface and a garment facing surface, the chassis extending in a longitudinal direction and having a first longitudinally extending side edge and a second longitudinally extending side edge;
moving a first combined leg cuff and barrier cuff structure in the machine direction, the first combined leg cuff and barrier cuff structure comprising:
a plurality of leg elastic members;
at least one barrier cuff elastic member; and
a cuff material encompassing the plurality of leg elastic members and the at least one barrier cuff elastic member; and
bonding the first combined leg cuff and barrier cuff structure to the body facing surface of the chassis with a seam bond between the cuff material and the body facing surface of the chassis such that at least one of the plurality of leg elastic members is disposed outboard of the first lateral side edge of the chassis,
wherein the seam bond divides the first combined leg cuff and barrier cuff structure into a first leg cuff and a first barrier cuff, the first leg cuff having a first leg cuff un-elasticized portion extending between the seam bond and the leg elastic member of the plurality of leg elastic members most proximate to the seam bond, the first leg cuff having a first leg cuff elasticized portion extending between the leg elastic member most proximate to the seam bond and a distal end of the first leg cuff, and the first barrier cuff having a first barrier cuff un-elasticized portion extending between the seam bond and the at least one barrier cuff elastic member that is most proximate to the seam bond,
wherein a length of the leg cuff is greater than a length of the barrier cuff, and
wherein a length of the first leg cuff un-elasticized portion is less than or equal to a length of first barrier cuff un-elasticized portion.

12. The article of claim 1, wherein the length of the first leg cuff un-elasticized portion is less than the length of the first barrier cuff un-elasticized portion by between about 5% and about 50%.

13. The article of claim 1, wherein the length of the first leg cuff un-elasticized portion is less than the length of the first barrier cuff un-elasticized portion by between about 10% and about 50%.

14. The article of claim 1, wherein the length of the first leg cuff un-elasticized portion is less than the length of the first barrier cuff un-elasticized portion by between about 15% and about 50%.

15. The article of claim 1, wherein the length of the first leg cuff un-elasticized portion is less than the length of the first barrier cuff un-elasticized portion by between about 15% and about 45%.

16. The article of claim 1, wherein the length of the first leg cuff un-elasticized portion is less than the length of the first barrier cuff un-elasticized portion by between about 20% and about 45%.
